(19) United States
(12) Patent Application Publication
(10) Pub. No.: US 2009/0292270 A1
(43) Pub. Date: Nov. 26, 2009

(54) Fastener Tab

(76) Inventors: Gbenga S. Showole, Fairfield, OH (US); Rolf Simm, Erfstadt (DE); Henry Cheng Na, Cincinnati, OH (US); Gary Dean LaVon, Liberty Township, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/469,906

(22) Filed: May 21, 2009

Related U.S. Application Data
(60) Provisional application No. 61/055,853, filed on May 23, 2008.
(60) Provisional application No. 61/055,860, filed on May 23, 2008.

(51) Int. Cl. A61F 13/58; A61F 13/60

(52) U.S. Cl. 604/390; 604/389

(57) ABSTRACT

A fastener tab includes a tab substrate having a first side and a second side. A first adhesive region is applied to an installation portion of the first side and a second adhesive region is applied to a fastening portion of the first side. The first and second adhesive regions are configured to provide different adhesion characteristics. A disposable absorbent article includes at least one fastener tab and may also include release and attachment substrates that releasably retain the second adhesive region for storage and use, respectively.

### CROSS REFERENCE TO RELATED APPLICATION

This application claims the benefit of U.S. Provisional Application Nos. 61/055,853 and 61/055,860, each filed on May 23, 2008, the substances of which are incorporated herein by reference.

### FIELD OF THE INVENTION

This invention relates to fastener tabs for disposable absorbent articles.

### BACKGROUND OF THE INVENTION

Disposable absorbent articles, such as diapers, training pants, incontinence garments, feminine hygiene garments, and the like typically include a self-fastening mechanism that allows the absorbent article to be fastened about the waist of a wearer. One type of self-fastening mechanism is a fastener tab. The fastener tab includes an installation portion that is fixed to an anchor region of the absorbent article (e.g., diaper chassis, including the back waist portion) and a fastening portion that is configured to be fastened to an attachment region of the absorbent article, which is sometimes executed as a landing zone, to hold the absorbent article in place about a wearer's waist. The installation portion may be permanently affixed to the anchor region. The fastening portion is configured to be capable of removal from a release surface (e.g., a release substrate surface) on which it is installed during the manufacture of the absorbent article. The fastening portion is then able to be attached to the attachment region.

It is desirable that the fastening portion be capable of being removed and refastened to the attachment region several times while still providing an acceptable strength of connection with the attachment region during use. Thus, a typical fastener tab must provide two different types of adhesion with respect to the absorbent article, a permanent connection via the installation portion and a removable and refastenable connection via the fastening portion. Often, the fastening portion includes a layer of some type of mechanical fastening means, such as, for example, hooks. Hooks require a mating element in the attachment region, which typically includes a separate fibrous layer of loops. The hook/loop systems, as well as other mechanical fastening systems, however, add avoidable costs and complexities to the manufacture of absorbent articles. As such, it is desirable to eliminate these costs in order to provide a low cost absorbent article, while retaining the refastenable connection between the fastener tab and the attachment region.

An alternative to using a mechanical fastening system would be to use a fastener tab consisting only of an adhesive. Such a system would not provide a cost advantage, however, in light of how typical fastener tabs are made. For instance, typical fastener tabs are made from a plastic substrate that includes a coating of a single type of adhesive that covers both the installation portion and the fastening portion. Because a single type of adhesive is used for both the installation and fastening portions, the adhesive is not singularly optimized for both the installation or fastening portions. As such, additional features are often added to the absorbent article to achieve the desired fastener tab functionality. For example, if an adhesive that allows for removal and refastening of the fastening portion is used, the installation portion may require additional measures, such as mechanical bonding, to provide a permanent bond with the absorbent article. If an adhesive that allows for a permanent bond of the installation portion is used, the fastening portion will not be refastenable without the use of special coating on the release surface and the attachment region. These measures add additional material and processing costs to the absorbent article. As such, it is desirable to eliminate these costs in order to provide a low cost absorbent article. Specifically, it is desirable to provide a fastener tab that is capable of being permanently connected to the anchor region of the absorbent article and refastenably connected to the attachment region of the absorbent article without resorting to the use of extra measures (e.g., mechanical bonding or release surface coatings).

In order to overcome the problems discussed above, it would be desirable to create a fastener tab consisting of a substrate with one or more adhesives that create a fastening portion having different adhesion characteristics than the installation portion, such that mechanical fasteners on the fastening portion and mechanical bonds securing the installation portion to the chassis are unnecessary. Because of the scale and equipment commonly used by the fastener tab suppliers, however, such would be cost prohibitive. As background, typically, fastener tabs are fabricated from large (e.g., 6-8 feet) rolls of tape substrate off site from the absorbent article lines. The rolls of tape substrate are made by applying a release agent to one side of a tape substrate and winding the tape substrate onto a roll. As the roll of tape substrate is unwound the other side of the substrate is coated with an adhesive (typically via a spray coater), forming a tape stock. The tape stock is then rewound into wide rolls and cut to customer specified widths prior to shipping. Finally, at the place of manufacture of the absorbent article, the tape is unwound, cut to length, and joined to the chassis of an absorbent article. The use of prefabricated fastener tabs adds material cost to the absorbent article because it requires the use of release agent on the side opposite the adhesive side of the tape substrate so that the tape substrate can be rolled up for shipment.

Creating the above-described desired fastener tabs having different adhesion characteristics would be cost prohibitive and impractical for a fastener tab supplier for multiple reasons. First, in order to continue to fabricate large tape stock rolls, the supplier would have to invest in a more precise adhesive applicator (e.g., transition from a spray coater to multiple slot coaters), which would be cost intensive. Second, highly precise cutting units would need to be used to cut the appropriate fastener tab roll sections from the larger tape stock roll, which would be cost intensive and executionally impractical (waste would be substantially higher in this process). Third, fastener tab suppliers can't switch over to making individual fastener tab rolls because it would require substantially the same time to fabricate a much smaller fastener tab roll as it does a large tape stock roll, or be more capital intensive to do so. Thus, production time and cost would increase. For all of these reasons it would be desirable to fabricate a fastener tab on-line, simultaneously with the absorbent articles themselves. Particularly, it would be desirable to avoid the need to use a release agent, and to have the flexibility to make slight adjustments to the adhesion characteristics of the fastener tab for a variety of reasons, including adjusting adhesion characteristics to 1) geographic climate variations where the absorbent article is made or where it will be used, 2) needs of the wearer, 3) size/surface area of the fastener tab, and 4) the needs of the care provider.

### SUMMARY OF THE INVENTION

Made in accordance with an embodiment of the present invention, a fastener tab includes a tab substrate having a first side and a second side. A first adhesive region is applied to an installation portion of the first side and a second adhesive region is applied to a fastening portion of the first side. The first and second adhesive regions are configured to provide different adhesion characteristics.

Made in accordance with an embodiment of the present invention, a fastener tab includes a tape substrate having a first side and a second side. A first adhesive is applied to an installation portion of the first side to form a first adhesive region and a second adhesive is applied to a fastening portion of the first side to form a second adhesive region. The first and second adhesive regions are configured to provide different adhesion characteristics.

A fastener tab in accordance with an embodiment of the present invention may include a fastening portion, an installation portion, and a grip region disposed along an outer edge of the fastening portion that is substantially free of adhesive. The fastening portion has a first adhesive region and the installation portion has a second adhesive region. The fastening portion may have a lower peel force than the installation portion. The first adhesive region may include a first adhesive and the second adhesive region may include a second adhesive. The first adhesive and second adhesive may be applied according to different patterns, such as, for example, a continuous coating, stripes, or discrete regions. The adhesives in the first and second adhesive regions may be applied to a nonwoven layer of the tab substrate.

An absorbent article in accordance with an embodiment of the present invention includes a backsheet having an exterior surface and an interior surface. The exterior surface includes an anchor region and an attachment region. The absorbent article also includes at least one fastener tab. The fastener tab comprises a tab substrate having a first side and a second side, a first adhesive region applied to an installation portion of the first side and a second adhesive region applied to a fastening portion of the first side. The first and second adhesive regions are configured to provide different adhesion characteristics. The fastener tab is fixed to the anchor region with the first adhesive region and the second adhesive region is releasably connected to a release surface of a release substrate.

### BRIEF DESCRIPTION OF THE DRAWINGS

FIG. 1 is a plan view of an interior surface of an absorbent article comprising a fastener tab constructed in accordance with one or more embodiments of the present invention.

FIGS. 2a-2c are cross section views of a fastener tab installed on an absorbent article in accordance with one or more embodiments of the present invention.

FIGS. 3a-3d are plan views of fastener tabs constructed in accordance with various embodiments of the present invention.

FIG. 4 is a fragmentary plan view of an absorbent article comprising a fastener tab constructed in accordance with one or more embodiments of the present invention.

FIG. 5 is a fragmentary perspective view of a process step in the fabrication of fastener tabs according to one or more embodiments of the present invention.

FIG. 6 is a schematic illustration of a fastener tab constructed in accordance with one or more embodiments of the present invention being tested for T-Peel strength according to a described test method.

FIGS. 7a and 7b are schematic illustrations of a fastener tab constructed in accordance with one or more embodiments of the present invention being tested for dynamic shear strength according to a described test method.

FIGS. 8a and 8b are schematic illustrations of a fastener tab constructed in accordance with one or more embodiments of the present invention being tested for shear hang time according to a described test method.

### DETAILED DESCRIPTION OF THE INVENTION

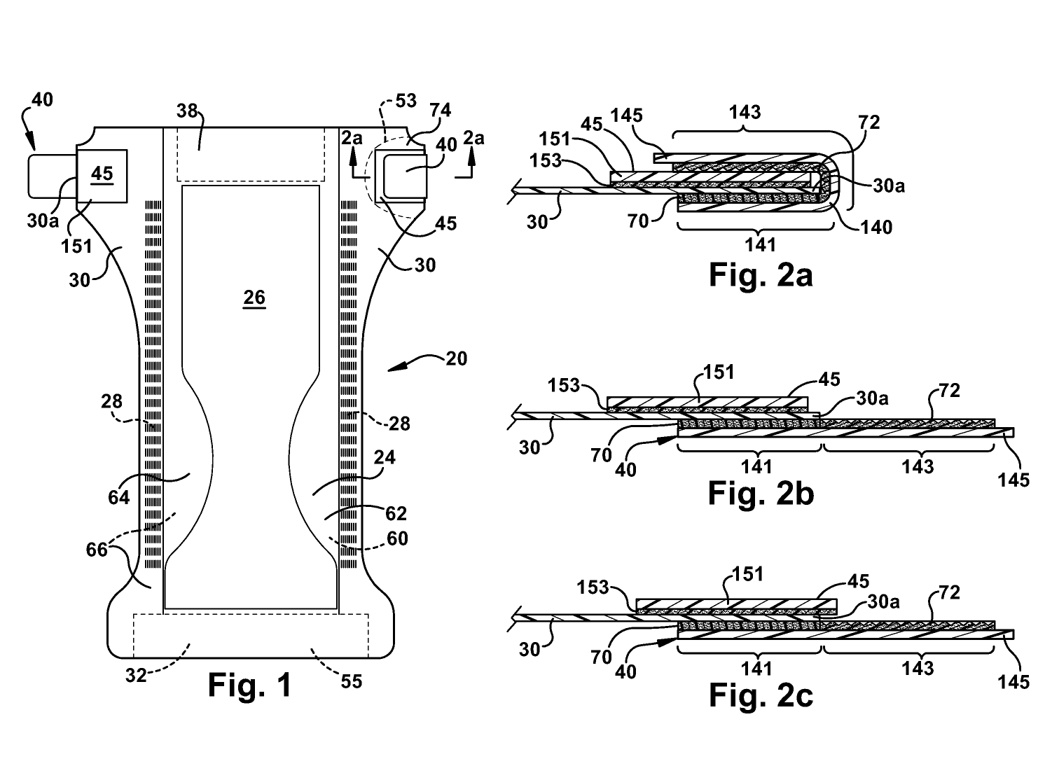

With reference to FIG. 1, an exemplary absorbent article 20 is shown. The absorbent article 20 generally comprises a liquid impermeable backsheet 24 and an absorbent core 26 fixed to the backsheet 24. The absorbent article 20 comprises a front waist region 32, a rear waist region 38, elasticized leg cuffs 28, ear flaps 30, at least one fastener tab 40, and an interior surface 62 and an exterior surface 60. The backsheet 24 is folded over at the edges of the absorbent article 20 and bonded to an elastic material to form the elasticized ear cuffs 28. Accordingly, the backsheet 24 of the absorbent article 20 has an exterior surface 66 and an interior surface 64. As will be described in more detail below, the fastener tab 40 is fixed to an anchor region 53 on the exterior surface 66 of the backsheet 24 in proximity to the ear flap 30. In other embodiments, the fastener tab 40 can be fixed to any appropriate region, on the interior surface 64 or the exterior surface 66 of the backsheet 24 of the absorbent article.

In FIG. 1, one of the fastener tabs 40 is shown pulled away from backsheet 24 and the other is shown folded over. At the time of manufacture, both of the fastener tabs 40 are folded over an edge 30a on the ear flap 30 and are releasably installed on a release surface 45 that is present on a release substrate 151 located on the interior surface 74 of the ear flap 30. In the described embodiment, the release surface 45 is formed by adhering a piece of release substrate 151 to the interior surface 74 of the backsheet in proximity to the ear flap 30. In other embodiments, the release surface 45 may be formed by treating the portion of material that a fastening portion 143 (FIG. 2a) of the fastener tab nests on (e.g., a portion of the backsheet 24, ear flaps 30, topsheet (not shown), etc.), such as by chemicals or heat, to create a releasable surface. Alternatively, the release surface 45 may not have any additional substrate or special surface treatment, in which case the fastening portion 143 of the fastener tab 40 is nested directly to an untreated surface of the backsheet 24, ear flaps 30, topsheet (not shown), etc.

When the absorbent article 20 is fastened to a wearer, the fastening portion 143 of the fastener tab 40 is deployed from the release surface 45 as shown in FIGS. 1, 2b, and 2c and attached to an attachment region 55 on the exterior surface 66 of the backsheet 24 to form leg openings (not shown) and secure the front waist 32 and rear waist 38 about the wearer's waist.

While an absorbent article 20 will be referenced in this description, it will be apparent to one of skill in the art that the fastener tab 40 described herein may be advantageously employed in connection with any number of absorbent articles 20 that utilize fastener tabs 40. Other exemplary absorbent articles that may be used with the fastener tabs 40 of the present invention include U.S. Pub. Nos. 2005/0171499, 2005/0203475, 2005/0288645, 2005/0288646, 2006/0264860, 2006/0264861, 2006/0271005, 2006/0293637, and 2006/0293638.

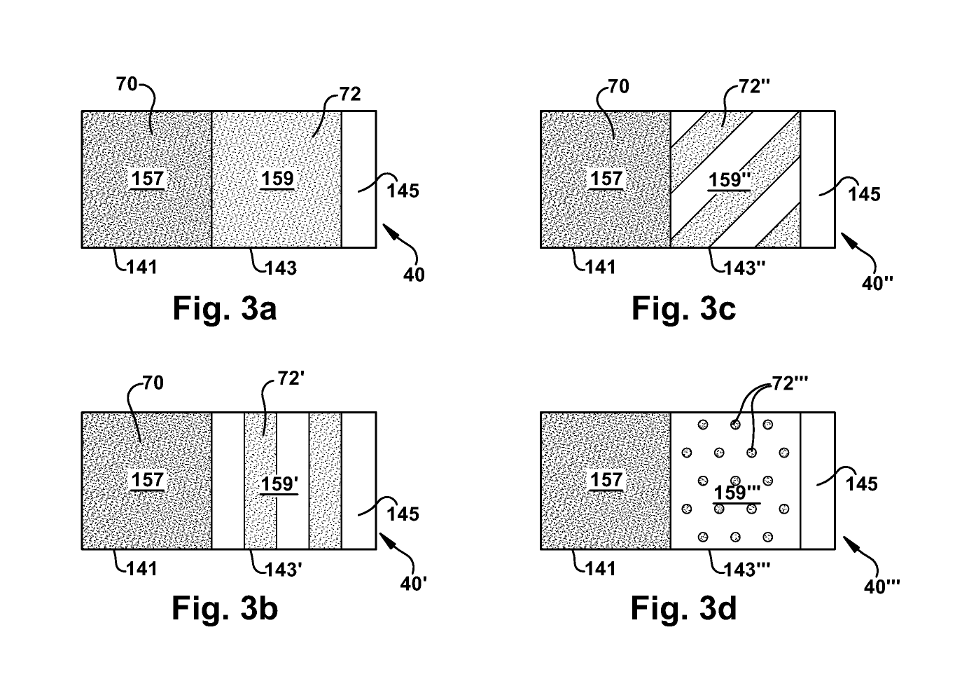

FIG. 2a is a cross section view of the fastener tab 40 folded over as installed or fixed on an ear flap 30 at the time of manufacture. FIG. 2b is a cross section view of the fastener tab 40 with the fastening portion 143 pulled away from the ear flap 30 and ready to be fitted to a wearer. The fastener tab 40 may comprise a tab substrate 140 having various adhesive regions of different adhesion characteristics(e.g., a first adhesive region 157 in the installation portion 141 and a second adhesive region 159 in the fastening portion 143). The first and second adhesive regions 157 and 159 can be seen best in FIGS. 3a-3d.

Many tab substrates 140 are known, many of which are laminates of various films and/or nonwovens and include at least one side that is a plastic film on which adhesive is applied. Tab substrates 140 are typically constructed to have a tensile strength of more than about 18 Newtons per centimeter (“N/cm”) and have a thickness of at least 50 microns. Nonwovens are often selected for an external layer of the tab substrate 140 because they present a soft, cloth-like texture. However, because nonwovens have holes or interstitial spaces between their fibers through which adhesive is prone to migrate, the tab substrate 140 includes a plastic film layer (not shown) on which adhesive (e.g., a first adhesive 70 and second 72) is applied. To construct such a laminated tab substrate 140, the nonwoven is typically glued to a plastic film layer and the plastic film layer is then coated with adhesive.

One exemplary tab substrate (not shown) includes two nonwoven, spun-bond fiber layers on either side of a melt-blown layer (not shown). In this embodiment, the various adhesives may be applied directly to a nonwoven tab substrate instead of a plastic film. The nonwoven to which the adhesive is applied has an air permeability of less than about 12 m³/m²/min. It is believed that an air permeability in this range provides sufficient resistance against adhesive migration.

As may best be seen in FIGS. 2b and 2c, the fastening tab 40 may have three portions: an installation portion 141, the fastening portion 143, and a grip portion 145. A first adhesive 70 is applied to the installation portion 141 to form a first adhesive region 157 (FIGS. 3a-3d) that fixes the installation portion to the ear flap 30 (or, alternatively, the backsheet 24). A second adhesive 72 is applied to the fastening portion 143 to form a second adhesive region 159 (FIGS. 3a-3d) that is configured to have different adhesion characteristics than the first adhesive region 157 of the installation portion 141 of the fastener tab 40. For example, the fastening portion 143 may have a T-Peel strength, also known as a 90° peel force, than the installation portion 141. The adhesion characteristics of the fastening portion 143 should remain in an acceptable range even after removal and refastening of the fastening portion 143 to the attachment region 55. As used herein, “adhesion characteristic” refers to one of any number of properties that may be provided by various adhesive materials and method of adhesive application. Three exemplary adhesion characteristics are T-Peel strength, dynamic shear, and tape shear hang time. Exemplary test methods for measuring each of these quantities are described in more detail below. The fastening portion 143 is configured to be releasably installed on the release surface 45 prior to use and refastenably affixed to the attachment region 55 (e.g., backsheet 24) in use. The installation portion 141 is permanently affixed to the ear flaps 30. The grip portion 145 is substantially free of adhesive and facilitates removal of the fastening portion from the release surface 45 by providing a tab that can be gripped by a user. In alternative embodiments (not shown), the tab is formed by folding over the edge of the fastening region 143.

The release surface 45 may be formed by, for example, a release substrate 151 that is adhered to an interior surface 74 of the ear flap 30 with release substrate adhesive 153. One exemplary release substrate 151 is an 80 micron thick plastic film coated with a release substrate adhesive 153 such as, for example, Bostik 2861. In the embodiment shown in FIGS. 2a and 2b the release substrate 151 is installed so that it does not overlap an edge 30a of the ear flap 30. In the embodiment shown in FIG. 2c the release substrate 151 overlaps the edge 30a of the ear flap 30 such that the release substrate adhesive 153 may be placed in contact with the second adhesive region 159 on the fastening portion 143. This configuration is commonly referred to as a Y bond configuration.

The difference in adhesion characteristics between the first adhesive region 157 on the installation portion 141 and the second adhesive region 159 on the fastening portion 143 may be achieved in any number of ways. For example, the fastener tab 40 shown in FIG. 3a includes a substantially continuous coating of a first adhesive 70 in the first adhesive region 157 and a substantially continuous coating of a second adhesive 72 in the second adhesive region 159, the second adhesive 72 being selected to enable the fastening portion 143 to have a lower T-Peel strength than that of the installation portion 141. The second adhesive region 159 may include a different type of adhesive than the adhesive in the first adhesive region 157. Alternatively, the second adhesive region 159 may include the same adhesive as the first adhesive region 157, but with a different basis weight. In an exemplary embodiment, the first adhesive 70 has an average T-Peel strength greater than about 10 N and the second adhesive 72 has an average T-Peel strength of about between 4-8 N. It is desirable to selectively configure the first and second adhesive regions 157 and 159 so that while they have different T-Peel strengths, dynamic shear and shear hang times are maintained above minimum acceptable levels. For example, a minimum dynamic shear of at least 10 N and a shear hang time of at least 10 minutes may be appropriate for some embodiments. FIG. 3a also shows the grip region 145 that is substantially free of adhesive.

The fastener tabs 40 shown in FIGS. 3b-3d have fastening portions 143′-143′″ with second adhesive regions 159′-159′″ that include the second adhesive 72′-72′″ applied in various patterns to achieve a different adhesion characteristic than in the first adhesive region 157. The various patterns are formed from discontinuous regions of the second adhesive 72′-72′″in the second adhesive regions 159′-159′″. In the described embodiments the pattern is formed by selectively applying the second adhesive 72′-72′″ to the tab substrate 140 according to the pattern so that in some parts of the second adhesive regions 159′-159′″, the tab substrate 140 is exposed, or substantially free of the second adhesive 72′-72′″. In other embodiments, the patterns may be formed by selectively de-activating parts of a continuous coating of adhesive (e.g., the second adhesive region 159) so that the pattern is formed from discontinuous regions of active adhesive separated by regions having de-activated adhesive. In embodiments with a patterned application of the second adhesive 72 in the second adhesive region 159, the same adhesive (e.g., the second adhesive 72) may be used in the first and second adhesive regions 157 and 159 to achieve different adhesion characteristics between the fastening portion 143 and the installation portion 141. In these instances, the difference in adhesion is driven by the reduced surface area of active adhesive in the fastening portion 143 as compared with the installation portion 141. Alternatively, the second adhesive region 159 may include a second adhesive 72 that is a different type of adhesive than the first adhesive 70 in the first adhesive region 157. FIG. 3b shows a second adhesive region 159′ with a second adhesive 72′ applied in a vertical stripe pattern. FIG. 3c shows a second adhesive region 159″ with a second adhesive 72″ applied in an offset stripe pattern. FIG. 3d shows a second adhesive region 159′″ with a second adhesive 72′″ applied in discrete regions. The discrete regions of the second adhesive 72′″ may be of any shape or free forms, such as, for example, dots, diamonds, squares, rectangles, or other geometric shapes.

In other embodiments the second adhesive region 159 on the fastening portion 143 is configured to fix some other fastening mechanism, for example, a mechanical fastening mechanism, such as a patch of hooked material that is configured to engage a fibrous layer of loops in the attachment region (not shown), to the fastener tab.

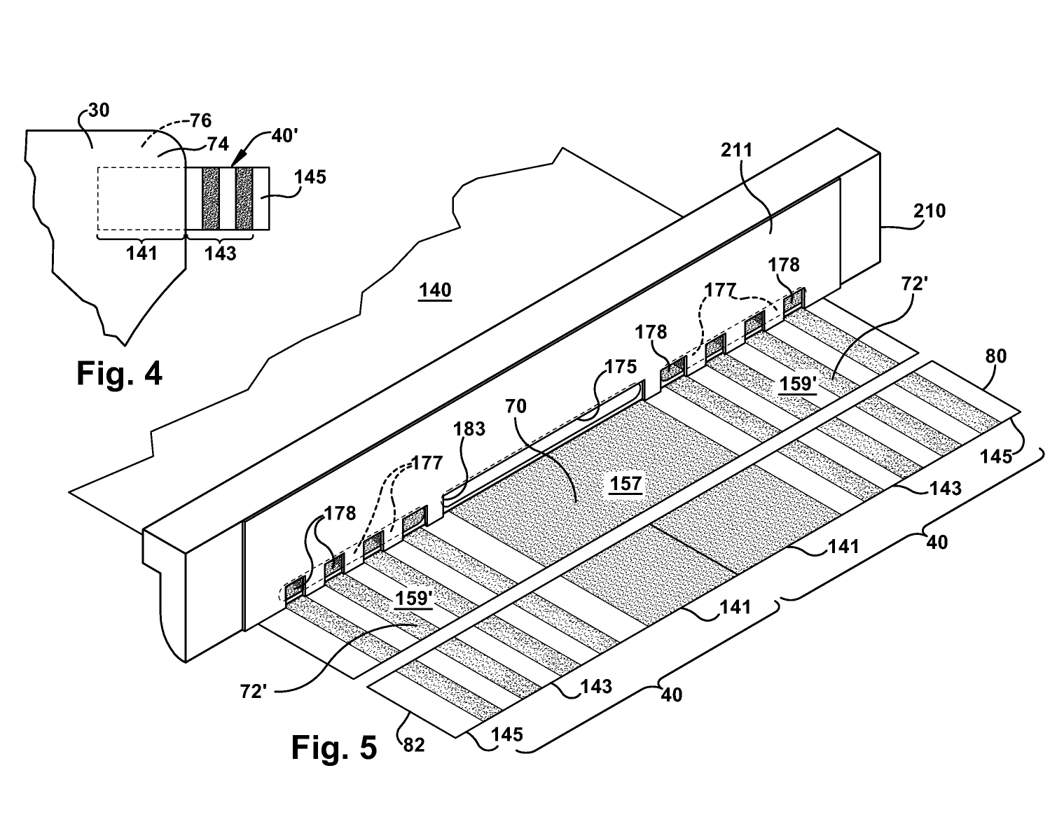

FIG. 4 is a fragmentary plan view of an ear flap 30 with the tab fastener 40′ as shown in FIG. 3b. The absorbent article 20 shown in FIG. 4 does not include a release substrate 151 (FIG. 2a) but rather uses the interior surface 74 of the ear flap 30 as a release surface (e.g., release surface 45). The installation portion 141 is shown in phantom as applied to the exterior surface 76 of the ear flap 30. It is believed that the type of adhesive used in the second adhesive region 159 on fastening portion 143 and/or the pattern in which the second adhesive 72′ is applied in the second adhesive region 159′ may be selected to eliminate the need for a separate release substrate (e.g., release substrate 151 in FIG. 2a) so that the fastening portion 143 may be releasably fastened directly to the ear flap 30 prior to use. In addition, proper selection of adhesion characteristics in the second adhesive region (e.g., 159′) may eliminate the need for a special release substrate (e.g., release substrate 151) to be used. Also, in these embodiments, the backsheet 24 may serve as the attachment region 55 (FIG. 1).

### Exemplary Test Methods

The various adhesion characteristics of a bond between an adhesive region (e.g. first adhesive region 157 and the second adhesive region 159) and the absorbent article 20 (e.g., the anchor region 53 and the attachment region 55) can be quantified in many ways. Three commonly measured qualities of the bond are, for example, T-Peel strength, dynamic shear, and shear hang time. These qualities can be measured by many different methods that are recognized in industry. For informational purposes, an exemplary method for measuring T-Peel strength, dynamic shear, and shear hang time are provided below.

For each of the sample preparations described below, the fastener tab 40, the anchoring region 53, and the attachment region 55 must be handled with care to avoid contact with hands, skin, or other contaminating surfaces. Clean sheets of untreated paper may be used to protect the surfaces of the fastening surfaces during the sample preparation.

### T-Peel Test

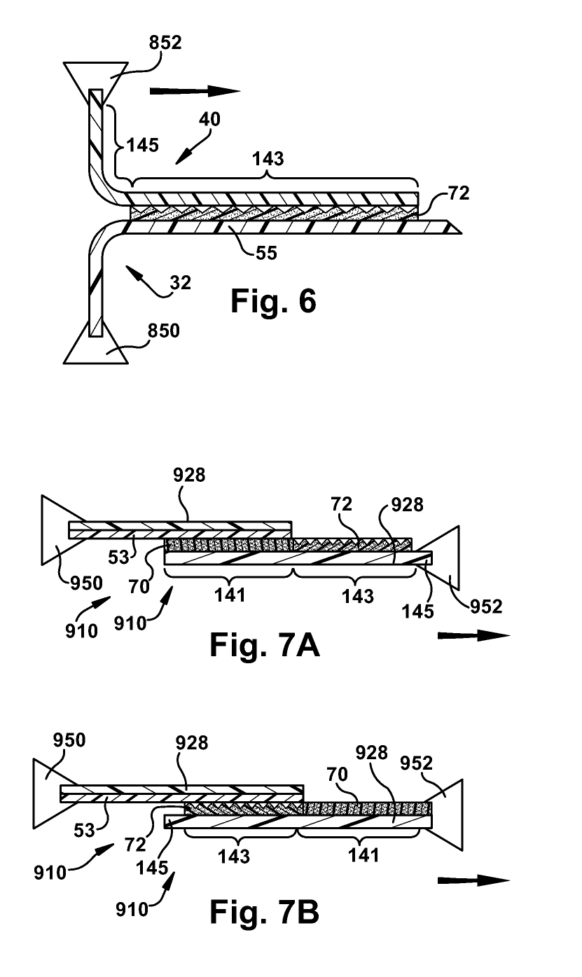

Referring now to FIG. 6, a cross section view of portion of a fastener tab 40 (e.g., the fastening portion 143 and the grip portion 145) and a sample attachment region 55 are shown installed in clamps 850 and 852. The clamps are part of a tensile tester (not shown) that is suitable for T-Peel strength testing. The following T-Peel test method is used to determine the T-Peel strength of the bond formed between the fastening portion 143 and the attachment region 55 and after fastening and/or refastening the fastening portion 143 to the attachment region 55.

Sample Preparation—The sample preparation for T-peel test will vary based on whether the fastener tab 40 and specifically the fastening portion 143 is available as a discrete web or is incorporated in a product. T-Peel forces referenced herein are in the context of the fastening portion 143 being tested after being bonding to the attachment region 55.

A skilled artisan should recognize that bonded specimens of other dimensions may be used in the T-Peel Method. The dimensions of the receiving and engaging members may vary from those listed above; however, the effective bonding area should be used to normalize the resultant T-Peel force recorded per inch of bonded width (i.e., the bonded width being the width of the bonded area measured substantially parallel to the grip width once the sample is mounted in the tensile tester).

Materials incorporated in a product: To perform the T-peel test, the fastener tab 40 material is cut from the product so as to isolate the fastening portion 143 and the attachment region 55, if possible. Removal of the materials from the product should be done to preserve the integrity of the materials (e.g., fastener tab 40, especially the fastening portion 143, and the attachment region 55 should not be permanently deformed or should not be debonded from each other). Before loading the samples for T-peel test, the fastening portion 143 and attachment region 55 should be separated approximately 1-5 mm to initiate the peeling if a grip portion 145 is not available. The front waist 32 portion of the sample including the attachment region 55 is the receiving sample, and the fastener tab 40 of the sample including the fastening portion 143 is the engaging sample. The receiving sample and engaging sample should each extend at least 5 millimeters beyond the bonded portion of the samples such that the proximal edge of the receiving sample and the proximal edge of the engaging sample can be easily placed in the test instrument's grips. If needed, an additional length of 2 mil PET film (not shown) may be attached to the proximal edges of the front waist 32 and the grip portion 145 using double sided tape. The T-peel test should be performed on the bonded materials as described in the method below. A skilled artisan should recognize that peel angle can affect the peel force. During peeling, the peel angle should be maintained around 90 degrees. Furthermore, if the fastening portion 143 or attachment region 55 are elastomeric, the fastening portion 143 or attachment region 55 must be backed with a similar sized sheet of 2 mil (0.05 mm) PET film (not shown) in order to prevent stretching of the tested substrate.

If the product is not pre-engaged, the materials are cut from the product and sample preparation would be similar to the method presented above for a sample in a film form. The average load calculated in peel force test should be normalized by the width of the fastener (in inches).

Refastened samples—Any of the above mentioned bonded samples may be refastened. The bonded sample is debonded using the tensile tester and following the Test Conditions for the T-Peel Test as provided for below (e.g., crosshead speed of 12 inches/minute). The fastening portion 143 and attachment region 55 are refastened in a configuration substantially similar to the configuration in which they were originally attached while avoiding wrinkles with only exception being that a fresh surface of the attachment region 55 is used. The refastened sample is rolled with a 4.5 pound (2 kg) HR-100 ASTM 80 shore rubber-faced roller. Two full strokes (i.e., back and forth) are applied to the sample at a speed of approximately 10 mm/sec (i.e., rolling should take approximately 40 seconds. After 1 minute of dwell time, the T-Peel Test is performed. This is the first refastened T-Peel force. This procedure may be repeated as needed to yield sequential refastened T-Peel forces (i.e., a second refastened T-Peel force, a third refastened T-Peel force, etc.).

Test Conditions—The T-Peel test method is performed in a controlled room at 22° C.+/−2° C. and RH 50% +/−10%. Suitable instruments for this test include tensile testers commercially available from Instron Engineering Corp., Canton, Mass. (e.g. Instron 5564) or from MTS Systems Corp., Eden Prairie, Minn. (e.g. Alliance RT/1 or Sintech 1/S). The following procedure illustrates the measurement when using the Instron 5564. The instrument is interfaced with a computer loaded with the Instron® Merlin™ Material Testing Software which controls the testing parameters, performs data acquisition and calculation, and provides graphs and data reports. The instrument is configured with a data acquisition speed of 50 Hz. Any resulting graphs are plotted using the Average Value (integral) setting on the instrument. A load cell is selected so that the forces to be measured will be between 10% and 90% of the capacity of the load cell or the load range used (e.g., typically, a 10 N to 100 N load cell). The instrument is calibrated to an accuracy of at least 1% and, ideally, less than 0.1% according to the manufacturer's instructions. The instrument has two grips: a stationary grip 850 and a movable grip 852. The grips 850, 852 used are wider than the sample; typically, 2 inch (5.08 cm) wide grips are used. The grips 850, 852 are air-actuated grips and are designed to concentrate the entire gripping force along a plane perpendicular to the direction of testing stress. The distance between the lines of the gripping force (i.e., gauge length) is set to 1″ (2.54 cm). The load reading on the instrument is zeroed to account for the mass of the fixture and grips. The bonded sample is mounted into the grips. The bonded sample is mounted so that the proximal edge of the receiving sample is in the movable grip and the proximal edge of the engaging sample is in the stationary grip. The bonded sample is mounted such that there is a minimum amount of slack in the receiving sample or engaging sample between the grips. The load cell is zeroed.

The receiving sample is separated from the engaging sample using a crosshead speed of 12 inches/min (305 mm/min). An average load is calculated as the average load between about 1″ (about 25 mm) and about 3.5″ (about 88 mm) displacement. For samples that do not meet the dimensions provided in the Sample Preparation, the average load is calculated from the loads acquired from the crosshead extension between about 25% to about 87.5% of the sample length. For example, if the sample is 6 inches long, the average load is calculated between about 1.5 inches and about 5.24 inches of crosshead extension. The average load is normalized to a width of 1″ (2.54 cm) as follows: normalized load=average load÷initial bond width in inches.

### Dynamic Shear Test Method

FIGS. 7a and 7b show a fastener tab 40 installed in clamps 950 and 952 for dynamic shear testing. This method is used to determine the shear strength of the bond formed between the fastener tab 40, specifically the installation portion 141, to the anchor region 53 and the fastener tab 40, specifically the fastening portion 143, to the attachment region 55. Additionally this method is used to determine the shear strength of the bond formed between the fastener tab 40, specifically the fastening portion 143, after refastening such that a bond has been broken and subsequently a new bond created with the fastening portion 143 and a fresh section of the attachment region 55. FIG. 7a shows a configuration in which the installation portion 141 is bonded to the anchor region 53 for testing. FIG. 7b shows a configuration in which the fastening portion 143 is bonded to the attachment region 55 for testing. The dynamic shear test method is performed in the same environmental conditions and with the same instrument as disclosed in the T-Peel Test.

Sample Preparation—The sample preparation for Dynamic Shear test will vary based on whether the material is available as a discrete web or is incorporated in a product. Dynamic shear forces referenced herein are in the context of the fastening portion being tested after being bonded to the attachment region.

To perform the dynamic shear test, the material is cut from the product so as to isolate the anchor region 53 or the attachment region 55 and the installation portion 141 or the fastening portion 143 respectively, if possible. Removal of the materials from the product should be done to preserve the integrity of the materials (e.g., anchor region 53 and installation portion 141 should not be permanently deformed or debonded). The anchoring region 53 is attached to a 2″×6″ stainless steel plate 928 to form an engaging sample. The installation portion 141 should have a distal edge that extends at least 5 millimeters from the bonded portion of the fastener tab 40 and anchor region 53 such that the distal edge can be easily inserted into the test instrument's grips. If needed, an additional length of 2 mil PET film (not shown) may be attached to the distal edge of the fastener tab 40 using double sided tape. The Dynamic Shear test should be performed on the bonded materials as described in the method below.

If the product is not pre-engaged, the materials are cut from the product and sample preparation would be similar to the method presented above for a sample in a film form.

The sample is rolled with a 4.5 pound (2 kg) HR-100 ASTM 80 shore rubber-faced roller. Two full strokes (i.e., back and forth) are applied to the sample at a speed of approximately 10 mm/sec. The resulting sample, called a bonded sample 910, is allowed to sit for 1 minute of dwell time.

Test Conditions—The Dynamic Shear test method is performed in a controlled room at 22° C. +/−2° C. and RH 50% +/−10%. The tensile tester is the same as used in the T-Peel test. A load cell is selected so that the forces to be measured will be between 10% and 90% of the capacity of the load cell or the load range used (e.g., typically, a 100 N to 250 N load cell). The instrument is calibrated to an accuracy of at least 1% and, ideally, less than 0.1% according to the manufacturer's instructions. The tensile tester has two grips: a stationary grip 950 and a movable grip 952. The grips are wider than the fastener tab 40 or anchor region 53 (e.g., typically, about 1 to about 2 inch wide (2.54-5.08 cm)). The grips 950, 952 are air-actuated grips and designed to concentrate the entire gripping force along a plane perpendicular to the direction of testing stress. Referring to FIG. 7a, the distal edge of the metal plate 928 affixed to the anchoring region 53 is mounted into the stationary grip 950. The distal edge of the fastener tab 40 is mounted into the movable grip 952. The bonded sample is to be mounted into the grips 950, 952 so that there is a minimum amount of slack and the load measured is less than 0.5 N. The distance between the lines of the movable grip 952 and the proximate edge of the bond site is about 1.3 inches (about 3.3 cm). The load reading on the instrument is zeroed.

The anchoring region 53 is separated from the installation portion 141 using a crosshead speed of 12 inches/min (305 mm/min) until the two samples are completely disengaged or one of the bonded samples 910 fails (e.g., the anchor region 53 tears, the fastener tab 40 tears, or the sample debonds at an interface other than of that between the anchoring region 53 and the installation portion 141).

If the bonded sample fails at any location other than the interface between the anchoring region 53 and the installation portion 141 prior to reaching a maximum load of at least 20 N/in², the data is to be discarded and another sample must be run using a backing material to prevent the sample form tearing and/or using a stronger double sided tape.

The Maximum Load is recorded and normalized to Newtons per inch² as follows: normalized load=measured load÷bonded area in inches squared.

### Shear Hang Time Test Method

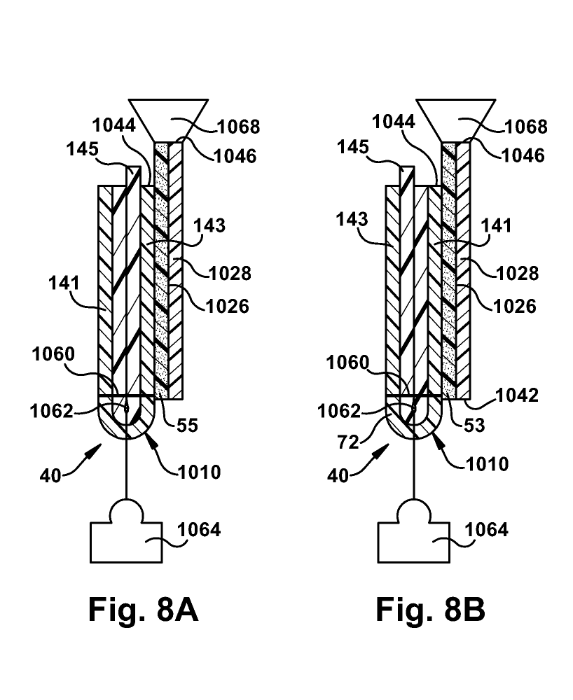

FIGS. 8a and 8b illustrate a sample fastener tab 40 installed on backing surface (e.g. an attachment region 55 in FIG. 8a and an anchor region 53 in FIG. 8b) and looped to hold a weight 1064 for shear hang time testing. This method is used to determine the shear resistance, measured in time, of the bond formed between the fastener tab 40, specifically the installation portion 141 to the anchor region 53 (FIG. 8b) and the fastener tab 40, specifically the fastening portion 143 to the attachment region 55 (FIG. 8a) when the bond is subjected to a load in controlled temperature environments. Additionally this method is used to determine the shear resistance, measured in time of the bond formed between the fastener tab 40, specifically the fastening portion 143, after refastening such that a bond has been broken and subsequently a new bond created with the fastening portion 143 and a fresh section of the attachment region 55, again when the bond is subjected to a load in controlled temperature environments. This test is derived from FINAT Test Method No. 8, the European Association for the Self Adhesive Tape Industry (AFERA) Test Method No. 4012, and ASTM-D Test Method No. 6463.

Sample Preparation—The sample preparation for Shear Hang Time test will vary based on whether the material is available as a discrete web or is incorporated in a product. Tape shear hang time results referenced herein are in the context of the installation portion being incorporated in the product at the anchor region.

For a fastener tab 40 having a fastening portion 143 or a installation portion 141, an attachment region 55 or anchor region 53 respectively is resized using cutting dies to create a rectangular sample with the dimensions of about 3.5 cm×about 7.5 cm (1.4″×3.0″). The adherend being either an attachment region 55 or anchor region 53 is backed with a like sized backing sheet of (polyethylene terephthalate) film or paper (not shown). The backing sheet must be positioned and sized so as to not interfere with the interface of the fastening portion 143 or installation portion 141 to the attachment region 55 or anchor region 53 respectively.

For the attachment region 55 or anchor region 53, an approximately 1.3 cm×2.54 cm (0.5″×1″) piece of an attachment region 55 or anchor region 53 is bonded in a face-to-face relationship to a similarly sized piece of double-sided tape 1026 (such as FT 239 available from Avery Denninson Corp., Painesville, Ohio or 9589 available from 3M, St. Paul, Minn.). The fastening portion 143 or installation portion 141 is to be wrinkle free. It should be appreciated that the fastening portion 143 or installation portion 141/double sided tape 1026 laminate can be created with larger sized materials and then resized to 1.3 cm×2.54 cm. The other side of the double side tape 1026 is bonded to a test panel 1028 having a proximal edge 1042 and a distal edge 1046. The double side tape 1026 is bonded adjacent the proximal edge 1042 of the test panel 1028. The test panel 1028 is ideally made from steel (ASTM A666 specification); alternately, the test panel 1028 may be made from a corrugated cardboard with a thickness of at least about 3-4 mm. The attachment region 55 or anchor region 53 is bonded onto the fastening portion 143 or installation portion 141 respectively. The bonded sample 1010 is then rolled with a 4.5 pound (2 kg) HR-100 ASTM 80 shore rubber-faced roller. Two full strokes (i.e., back and forth) are applied to the sample 1010 at a speed of approximately 5 mm/sec. The bonded area should be approximately 2.54 cm×1.3 cm (0.5″×1.0″).

A skilled artisan should recognize that bonded specimens of other dimensions may be used in the Shear Hang Time Test Method. The dimensions of the receiving and engaging members may vary from those listed above. However, if the bonded area exceeds approximately 2.54 cm×1.3 cm (0.5″×1.0″), the sample should be resized to yield a bonded area of 2.54 cm×1.3 cm (0.5″×1.0″).

Materials incorporated in a product: To perform the tape shear hang time test, the material is cut from the product so as to isolate the attachment region 55 or anchor region 53 and the fastening portion 143 or installation portion 141 respectively, if possible. However, if the attachment region 55 or anchor region 53 and/or the fastening portion 143 or installation portion 141 respectively are joined to other materials in a face-to-face configuration, the face-to-face configuration between the attachment region 55 or anchor region 53 and the other material or the fastening portion 143 or installation portion 141 respectively and the other material should be maintained. Removal of the materials from the product should be done to preserve the integrity of the materials (e.g., the attachment region 55 or anchor region 53 and the fastening portion 143 or installation portion 141 respectively should not be permanently deformed and should not be debonded from each other). The fastening portion 143 or installation portion 141 should have a distal edge 1044 that extends at least 50 millimeters from the bonded portion of the attachment region 55 or anchor region 53 and the fastening portion 143 or installation portion 141 respectively such that the distal edge can be easily be folded over to form a loop 1062. If the distal edge does not extend at least 50 mm, an additional length of 2 mil PET film (not shown) may be attached to the distal edge 1044 using double sided tape. The shear hang test should be performed on the bonded materials as described in the method below.

If the product is not pre-engaged, the materials are cut from the product and sample preparation would be similar to the method presented above for a sample in a film form.

When determining the tape shear hang time of the fastening portion 143 to the attachment region 55 the bonded sample is rolled with a 4.5 pound (2 kg) HR-100 ASTM 80 shore rubber-faced roller. Two full strokes (i.e., back and forth) are applied to the sample at a speed of approximately 5 mm/sec. The sample is allowed to sit for 1 minute of dwell time.

Test Conditions—The bonded sample 1010 is prepared at ambient room conditions (e.g., 22° C.+/−2° C. and RH 50% +/−10%). The bonded sample 1010 is brought into a temperature chamber immediately prior to the commencement of testing. The time between introduction of the bonded sample 1010 into the temperature chamber and commencement of testing is to be less than 5 minutes. The test is conducted in a 100° F. controlled temperature chamber or oven (37.5° C. +/−2° C.). Suitable instruments for this test are the RT10 or RT30 available from ChemInstruments Inc, Fairfield, Ohio or any apparatus having a rack or jig capable of holding a test plate within 0° to 2° of vertical. The time is measured by an automated timer capable of reading to the nearest minute.

FIGS. 8a and 8b are cross-sectional views of the bonded sample 1010 in a test apparatus. The distal edge 1044 of the fastening portion 143 or installation portion 141 is folded onto itself and affixed with a staple 1060 to form a loop 1062. The distal edge 1046 of the attachment region 55 or anchor region 53 is placed into a rack 1068 so that the fastening portion 143 or installation portion 141 hangs downwards. The 1 kg weight 1064 is attached to the looped end of the fastening portion 143 or installation portion 141. The timer is started once the weight 1064 hangs freely from the fastening portion 143 or installation portion 141. The time required for debonding of the fastening portion 143 or installation portion 141 and the attachment region 55 or anchor region 53 is recorded (i.e., the fastening portion 143 or installation portion 141 separates and falls from the attachment region 55 or anchor region 53). The test can be manually stopped if the sample remains bonded beyond a prescribed time period.

If the bonded sample fails at a time less than specified with this disclosure for some reason other than separation of the interface between the fastening portion 143 or installation portion 141 with the attachment region 55 or anchor region 53 respectively (e.g., the fastening portion 143 or installation portion 141 tears, attachment region 55 or anchor region 53 tears, or the sample debonds at an interface other than of that between the fastening portion 143 or installation portion 141 and the attachment region 55 or anchor region 53 respectively, the data is discarded and another sample must be run using a backing material to prevent the sample from tearing and/or using a stronger double sided tape to prevent separation at interfaces other than between the fastening portion 143 or installation portion 141 and the attachment region 55 or anchor region 53 respectively.

Turning now to FIG. 5, exemplary process steps for manufacturing fastener tabs 40 that have first and second adhesive regions 157 and 159 with different adhesion characteristics are illustrated. Tab substrate 140 is passed under a slot coater 210 through which one or more types of adhesives (e.g., a first adhesive 70 and a second adhesive 72) are pumped. In the example illustrated in FIG. 5, the first adhesive 70 is applied to the first adhesive region 157 through a first nozzle 175 and the second adhesive 72′ is applied to the second adhesive region 159 through a pair of nozzles 177. The first adhesive 70 may be the same as the second adhesive 72 or the first and second adhesives may be different, as discussed above.

To achieve the striped pattern shown in the second adhesive region 159′, a shim 211 is placed on the slot coater 210 that blocks a portion of the nozzles 177. The shim 211 includes a central opening 183 corresponding roughly to the size and shape of the first nozzle 175. The shim also includes holes 178 that are aligned in front of the nozzles 177 and through which the second adhesive 72 flows to create the striped pattern of adhesive. The use of the shim 211 on the slot coater 210 enables the specific pattern of adhesive to be varied by a simple change of shims. It will be apparent to one of skill in the art that other patterns of adhesive (e.g., the first and second adhesives 70 and 72) can be achieved in the first or second adhesive regions 157 and 159 by varying the size, shape, or configuration of the shim 211, as well as the flow of adhesive through the first and second nozzles 175 and 177.

The grip region 145 can be seen on either side edge 80 and 82 of the processed tab substrate 140. If the grip region 145 is not desired, the second nozzles 177 may direct the flow of adhesive to the side edges 80 and 82 of the tab substrate 140.

The substrate 140 shown in FIG. 5 is approximately twice as wide as the length of a fastener tab 40. As can be seen in FIG. 5, two fastener tabs 40 are created from each lateral slice of tab substrate. One way to process the coated substrate to create fastener tabs 40 is to longitudinally (in the direction of the flow of substrate under the coater) slit the substrate and then laterally (perpendicular to the flow of substrate under the coater) cut the slit substrate to the desired tab width. While the segmented slot coater shown in FIG. 5 is well suited for applying first and second adhesives 70 and 72 with differing adhesion characteristics to adjacent regions of a substrate, other methods such as, for example, spraying, gravure, or flexographic adhesive application may be used.

While any number of substrates, laminates, films, and adhesives can be used in the practice of the present invention, the following materials are considered to be suitable examples. The fastener tab substrate 140 may be unglued Avery fastening tape (3-4 mil polypropylene), unglued Clopay tape (3-4 mil polyethylene), unglued Clopay tape (2-3 mil) laminated to 10 gsm nonwoven substrate, 78 gsm nonwoven (SMS and SMMS) from PGI, or a lamination of 66 gsm nonwoven (RKW)/50 gsm spunbond nonwoven (RKW)/cPP Plastic (80 micron) (Novel). The release substrate 151 may be unglued Avery release substrate (3-4 mil polypropylene), unglued Clopay tape (2 mil) silicone coated), or cPP plastic film (50 or 80 micron)(Novel). Adhesives that can be used as substrate adhesive 153 or in the first or second adhesive regions 157 and 159 can be Avery, hot melt, Fuller 1358LO, Bostik 2861, NSC 526, or NSC 395c.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A fastener tab comprising:
a tab substrate having a first side and a second side;
a first adhesive region on an installation portion of the first side; and
a second adhesive region on a fastening portion of the first side;
wherein the first adhesive region has a different adhesion characteristic than the second adhesive region.

2. The fastener tab of claim 1 wherein the first adhesive region comprises a first adhesive and the second adhesive region comprises a second adhesive.

3. The fastener tab of claim 1 comprising a grip portion disposed along an outer tab edge of the fastening portion, the grip portion being substantially free of adhesive.

4. The fastener tab of claim 1 wherein the adhesion characteristic of the second adhesive region when bonded to an attachment region on an absorbent article provides an average T-Peel strength of about between 4-8 Newtons.

5. The fastener tab of claim 1 wherein at least one of the first and second adhesive regions comprises a pattern of discontinuous regions of active adhesive on the tab substrate in the region.

6. The fastener tab of claim 4 wherein the peak T-Peel strength is between approximately 4-10 Newtons when the second adhesive region is bonded to the attachment region.

7. The fastener tab of claim 1 wherein the second adhesive region comprises an adhesive having a lower basis weight of adhesive than the basis weight of adhesive in the first adhesive region.

8. The fastener tab of claim 2 wherein the first adhesive is different than the second adhesive.

9. The fastener tab of claim 2 wherein at least one of the first and second adhesives is applied to form a substantially continuous coating of adhesive.

10. The fastener tab of claim 5 wherein the pattern on the at least one of the first and second regions comprises generally stripe shaped discontinuous areas of active adhesive.

11. The fastener tab of claim 5 wherein the pattern is formed by selectively applying the adhesive in the at least one of the first and second adhesive regions to form discontinuous areas of active adhesive separated by areas of exposed tab substrate.

12. The fastener tab of claim 5 wherein the pattern is formed by applying a substantially continuous coating of adhesive in the at least one of the first and second adhesive regions and then selectively de-activating areas of adhesive to form discontinuous areas of active adhesive separated by areas of de-activated adhesive.

13. The fastener tab of claim 5 wherein at least one of the first and second patterns applies adhesive in discrete regions of adhesive within the first or second adhesive regions.

14. The tab fastener of claim 1 wherein the substrate comprises at least one nonwoven layer.

15. The tab fastener of claim 14 wherein the first and second adhesive regions comprise adhesives that are applied to the nonwoven layer.

16. The tab fastener of claim 1 wherein the dynamic shear of the second adhesive region when bonded to an attachment region on an absorbent article is greater than approximately 10 Newtons.

17. The tab fastener of claim 1 wherein the dynamic shear of the first adhesive region when bonded to an anchor region on an absorbent article is greater than approximately 10 Newtons.

18. The tab fastener of claim 1 wherein the shear hang time of a one kilogram weight fixed to the first adhesive region bonded to an anchor region on an absorbent article is greater than approximately ten minutes.

19. The tab fastener of claim 1 wherein the shear hang time of a one kilogram weight fixed to the second adhesive region bonded to an attachment region on an absorbent article is greater than approximately ten minutes.

20. An absorbent article comprising:
a chassis comprising an exterior surface joined with an interior surface, the exterior surface including an anchor region and an attachment region, the anchor region and attachment region disposed relative to one another such that the anchor region is placed proximate the attachment region during use of the absorbent article; and
a fastener tab comprising a tab substrate having a first side and a second side, the fastener tab further comprising a first adhesive region on an installation portion of the first side and a second adhesive region on a fastening portion of the first side, wherein the first and second adhesive regions are configured to provide different adhesion characteristics, the fastener tab being fixed to the anchor region with the first adhesive region and the second adhesive region being releasably and selectively connectable to a release surface on the interior surface of the chassis and an attachment region on an exterior surface of the chassis.
